(19) United States
(12) Patent Application Publication
(10) Pub. No.: US 2023/0380525 A1
(43) Pub. Date: Nov. 30, 2023

(54) NANOPARTICLES FOR USE IN ANTI PATHOGENIC APPLICATIONS

(71) Applicant: PHARM 2 FARM LIMITED, London (GB)

(72) Inventors: Gareth Wynn Vaughan CAVE, London (GB); Karen DAVIES, Nottingham (GB)

(73) Assignee: PHARM 2 FARM LIMITED, London (GB)

(21) Appl. No.: 18/032,493

(22) PCT Filed: Oct. 21, 2021
(86) PCT No.: PCT/GB2021/052733

§ 371 Date: Apr. 18, 2023

(30) Foreign Application Priority Data
Oct. 22, 2020 (GB) 2016813.4

(51) Int. Cl. A41D 13/11; A01N 25/28; A01N 59/20; D01D 5/098

(52) U.S. Cl. CPC A41D 13/1192; A01N 25/28; A01N 59/20; D01D 5/0985; B82Y 30/00

(57) ABSTRACT

A dispersible nanoparticle (200) for use in anti-pathogenic applications is presented. The dispersible nanoparticle has a core (210) made of a metal or a metal alloy compound. The core (210) is coated with at least one of a carboxylic acid and a water soluble polymer (220). Also presented is a membrane coated with the dispersible nanoparticles and a corresponding method of coating the membrane. The membrane may be used is various products including a face mask and an air filter for use in an air conditioning unit.

### FIELD OF THE DISCLOSURE

The present disclosure relates to nanoparticles for use in anti-pathogenic applications. In particular, the present disclosure relates to dispersible metal oxide, metal hydroxide, metal-alloy oxide, or metal-alloy hydroxide nanoparticles having anti-viral, anti-bacterial or anti-parasitic properties.

### BACKGROUND

Human infections may occur when an infectious agent (pathogen) such as a virus, a bacteria or a parasite enters the body, for instance through respiratory tracks. Although the human body can defend itself against bacterial, viral and parasitic invasions, virulent agents are likely to generate diseases in the host after infection. When the infectious agent in question is particularly contagious this can lead to a global outbreak. The recent pandemics of H1N1, SARs and SARs-COV-2 have shown that proactive measures such as hand washing, and adoption of face masks can help slow down or even prevent human-to-human transmission of infectious agents. However, the infectious agent can still survive on a mask, in which case the mask itself can become a vector of infection.

Metallic materials are known to display anti-pathogen properties. Several studies have reported the antimicrobial and antiviral properties of copper over a variety of pathogens. In particular copper nanoparticles can be used to inhibit influenza virus and coronavirus. A. A. Cortes and J. M. Zuñiga, The use of copper to help prevent transmission of SARS-Coronavirus and Influenza viruses. A general Review, Diagnostic Microbiology & Infectious Disease (2020), littps://doi.org/10.1016/j.diagrnicrobio.2020.115176.

However, metal nano-particles are not readily dispersible and may vary in size. There is therefore a need for dispersible nanoparticles with anti-pathogen properties for use in anti-viral, anti-bacterial or anti-parasitic applications.

### SUMMARY OF THE DISCLOSURE

According to a first aspect of the disclosure, there is provided a dispersible nanoparticle comprising a core made of a metal or a metal-alloy compound, wherein the core is coated with at least one of a carboxylic acid and a water soluble polymer.

Optionally, the core further comprises an oxygen-based compound.

Optionally, the metal compound comprises zinc or a lanthanide. For example, the lanthanide may be cerium.

Optionally, the metal-alloy compound comprises copper.

Optionally, the oxygen-based compound comprises one of a carbonate ion, a nitrate ion, a nitrite ion, or a hydroxide ion.

Optionally, the carboxylic acid coating comprises at least one of an amino acid, and a fatty acid, and the water soluble polymer coating comprises at least one of a branched polymer, a sugar polymer, a hydrogel and ethylene glycol or polyethylene glycol.

Optionally, the dispersible nanoparticle has a diameter of less than about 100 nm. For instance, the diameter may range between about 5 nm and 60 nm. For example, the diameter may be about 50 nm.

According to a second aspect of the disclosure, there is provided a nanoparticle dispersion comprising a concentration of dispersible nanoparticles according to the first aspect of the disclosure.

Optionally, the nanoparticle dispersion is an aqueous dispersion.

Optionally, the concentration of nanoparticles ranges between about five percent and about fifteen percent of nanoparticles.

Optionally, the nanoparticle dispersion comprises at least one of copper, copper oxide, copper hydroxide, zinc oxide, zinc hydroxide, cerium oxide and cerium hydroxide nanoparticles. These nanoparticles may also be dopped. For instance cerium may be dopped with copper to form a cerium nanoparticle doped with copper.

Optionally, the nanoparticle dispersion is an anti-pathogenic solution having at least one of an anti-viral, an anti-bacterial, and an anti-parasitic property. For instance, the anti-viral property may inhibit or kill a target virus or group of viruses. Similarly the anti-bacterial property may inhibit or kill a target bacterium or group of bacteria, and the anti-parasitic property may inhibit or kill a target parasite or group of parasites.

Optionally, the nanoparticle dispersion comprises a polymer network to form a gel. For instance, the gel may be a branched polymer or a hydrogel.

The options described with respect to the first aspect of the disclosure are also common to the second aspect of the disclosure.

According to a third aspect of the disclosure, there is provided a membrane coated with the dispersible nanoparticles according to the first aspect of the disclosure.

Optionally, the membrane is treated with the nanoparticle dispersion according to the second aspect of the disclosure. For example, the membrane may be treated by dipping the membrane in the dispersion or spraying the membrane with the dispersion.

Optionally, the membrane is adapted to filter a volume of air. For example, the membrane may be designed to trap target particles having a given size, including pathogen such as bacteria, viruses, or parasites.

Optionally, the membrane is made of a non-woven polymer material. For example, the non-woven polymer material may be a melt blown material.

Optionally, the membrane is an anti-pathogenic membrane having at least one of an anti-viral, an anti-bacterial and an anti-parasitic property. For instance, the anti-viral property may inhibit or kill a target virus or group of viruses. Similarly the anti-bacterial property may inhibit or kill a target bacterium or group of bacteria, and the anti-parasitic property may inhibit or kill a target parasite or group of parasites.

The options described with respect to the second aspect of the disclosure are also common to the third aspect of the disclosure.

According to a fourth aspect of the disclosure, there is provided a product comprising a membrane according to the third aspect of the disclosure. For instance, the product may be a protective device that includes the membrane, such as a face mask. The product may also be a piece of clothing made at least in part of the membrane. Alternatively the product may be a filter included in a mechanical device such as an air conditioning unit.

Optionally the product is a face mask. For example, the face mask may be a respirator designed to protect the wearer from breathing in hazardous substance such as pathogens. For instance, the face mask may be a medical mask such as a surgical mask to be worn by health professionals.

Optionally, the membrane is sandwiched between an inner layer and an outer layer.

Optionally, the product comprises an additional membrane provided between the inner layer and the outer layer.

Optionally, the product is an air filter for use in an air conditioning unit, the membrane being part of the air filter.

Optionally, the product is a piece of clothing and wherein the membrane is made of textile or fabric material. For example, the piece of clothing may be a scarf that has been sprayed with the nanoparticle dispersion to protect against pathogens.

The product according to the fourth aspect of the disclosure may comprise any of the features described above in relation to the membrane according to the third aspect of the disclosure.

According to a fifth aspect of the disclosure, there is provided a method of coating a membrane, the method comprising providing a nanoparticle dispersion according to the second aspect of the disclosure, providing a membrane, dipping the membrane in the dispersion or spraying the membrane with the dispersion or printing the dispersion onto the membrane, and heating the membrane to bind the nanoparticles to its surface. For instance, infrared light may be used to heat the membrane and nanoparticles, so as to embed the nanoparticles into the surface of the membrane.

The fifth aspect may share features of the first, second, and third aspects of the disclosure as noted above and herein.

According to a sixth aspect of the disclosure, there is provided a liquid product for marking a substrate, wherein the liquid product comprises a nanoparticle dispersion according to the second aspect and a colorant comprising at least one of a dye and a pigment.

Optionally, the liquid product is an ink.

Optionally, the liquid product is a paint.

The sixth aspect may share features of the first and second aspects of the disclosure as noted above and herein.

### DETAILED DESCRIPTION

The disclosure is described in further detail below by way of example and with reference to the accompanying drawings, in which:

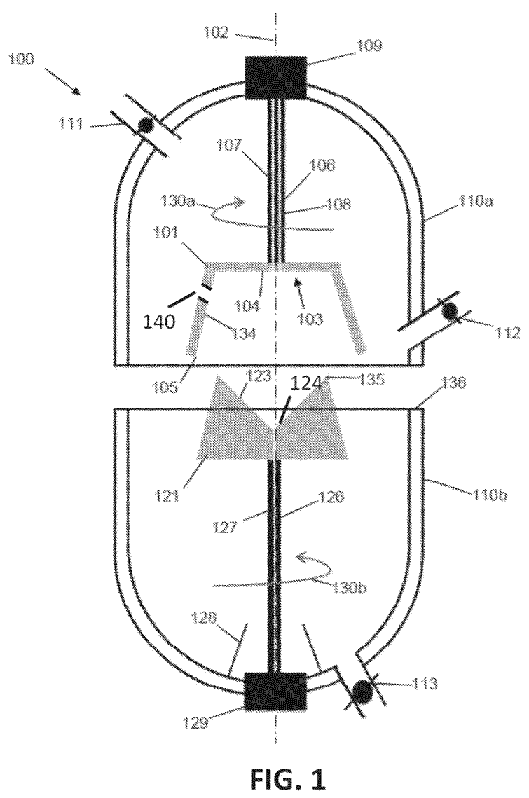

FIG. 1 is a diagram of a spinning disc reactor;

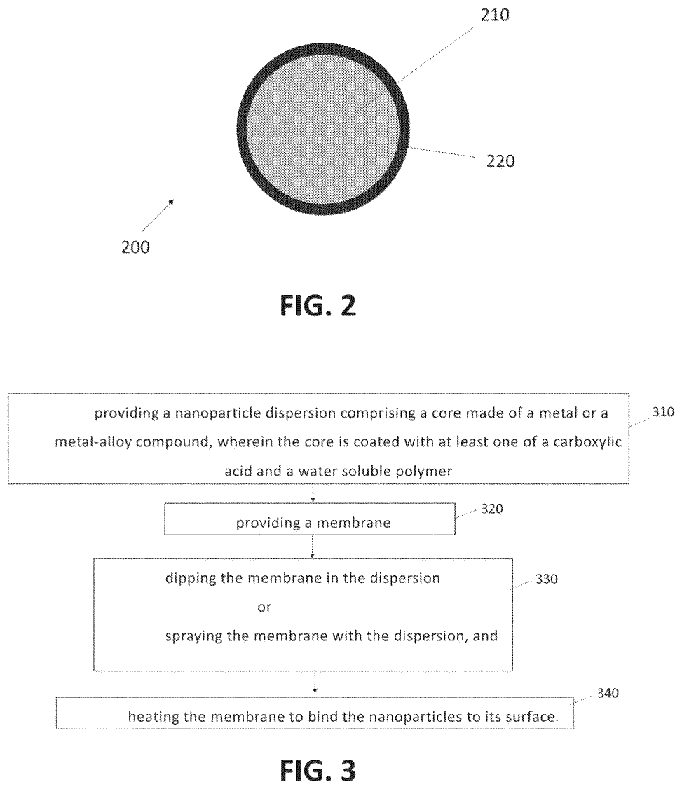

FIG. 2 is a schematic representation of a nanoparticle according to the disclosure;

FIG. 3 is a flow chart of a method for coating a membrane;

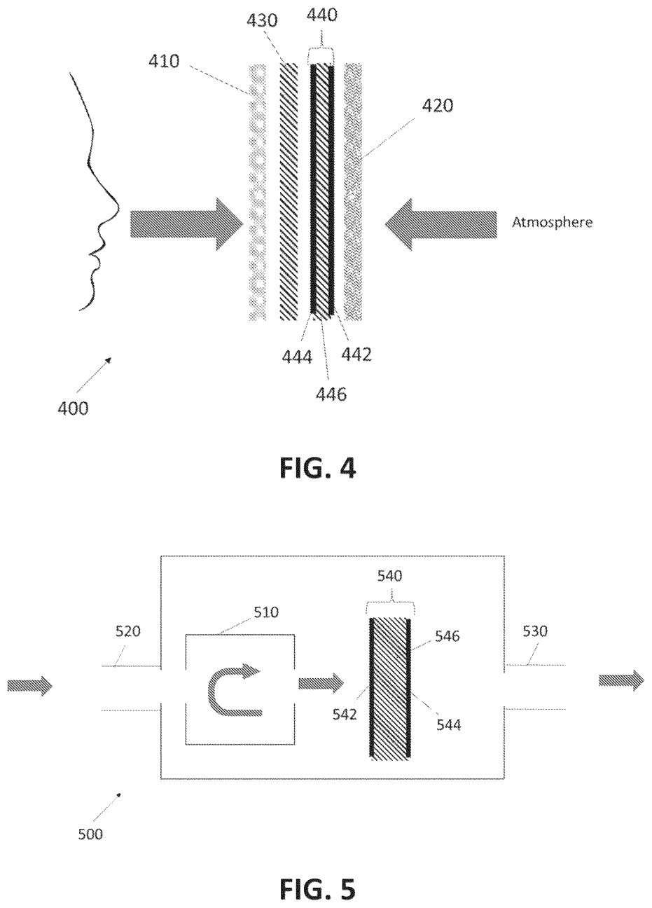

FIG. 4 is an exploded cross section of a face mask comprising a membrane coated with nanoparticles according to the method of FIG. 3;

FIG. 5 is a diagram of an air conditioning unit provided with a filter comprising a nanoparticle coated membrane;

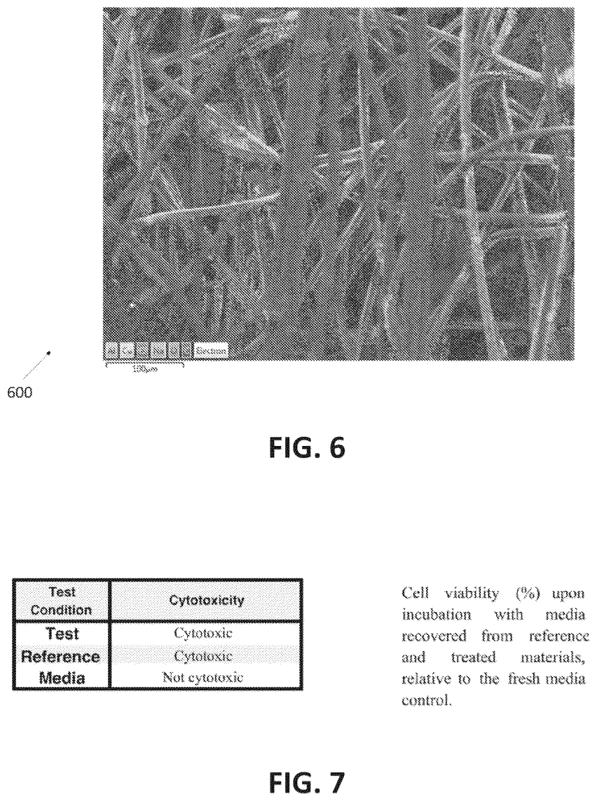

FIG. 6 is a transmission electron microscope image of a melt blown membrane treated with lysine-coated copper-oxide nanoparticles;

FIG. 7 is a cytotoxicity table;

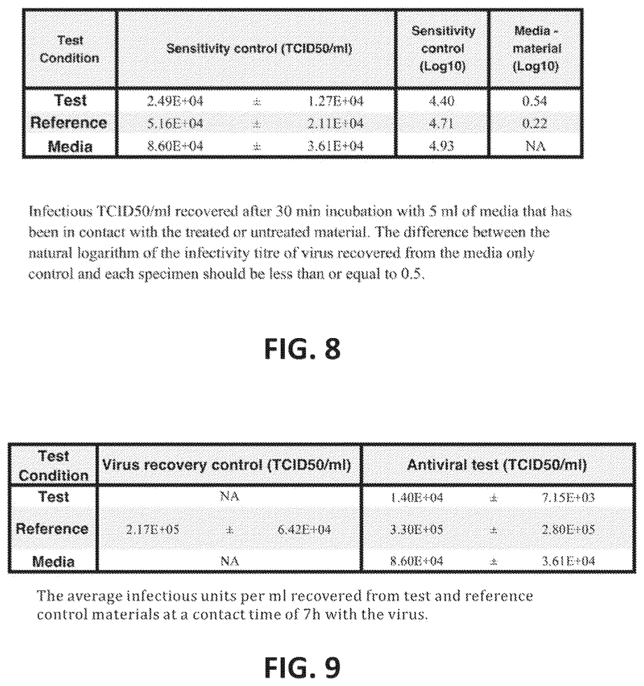

FIG. 8 is a sensitivity control table;

FIG. 9 is a table listing the average infectious units per ml recovered from test and reference control materials at a contact time of 7 h with influenza A virus;

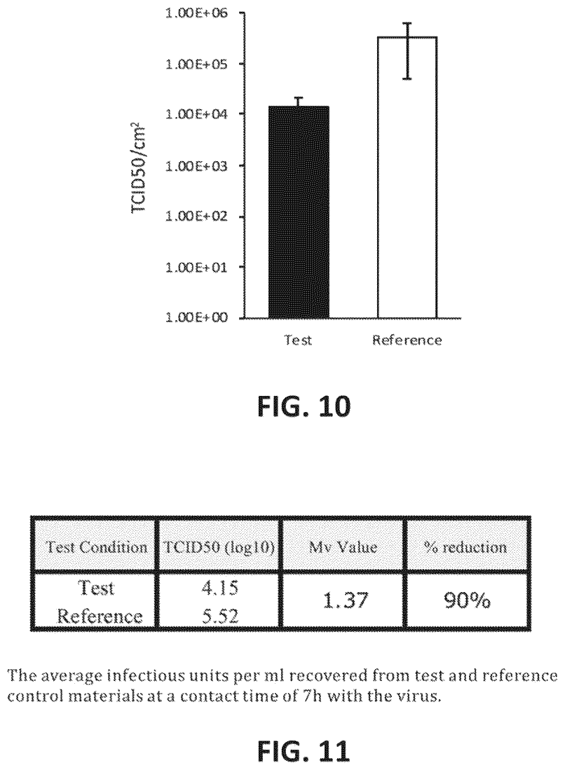

FIG. 10 is a graph showing the mean TDIC50/ml values for influenza A virus following a contact time of 7 h with test and reference control materials;

FIG. 11 is a table showing the antiviral activity of the treated membrane;

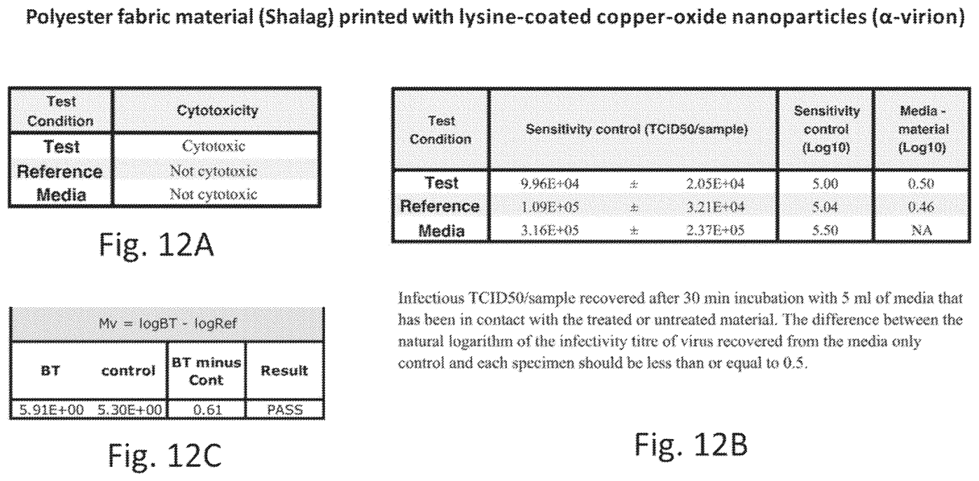

FIG. 12 shows a cytotoxicity table (A), a sensitivity control table (B) and a reduction table (C) for a polyester material printed with α-virion;

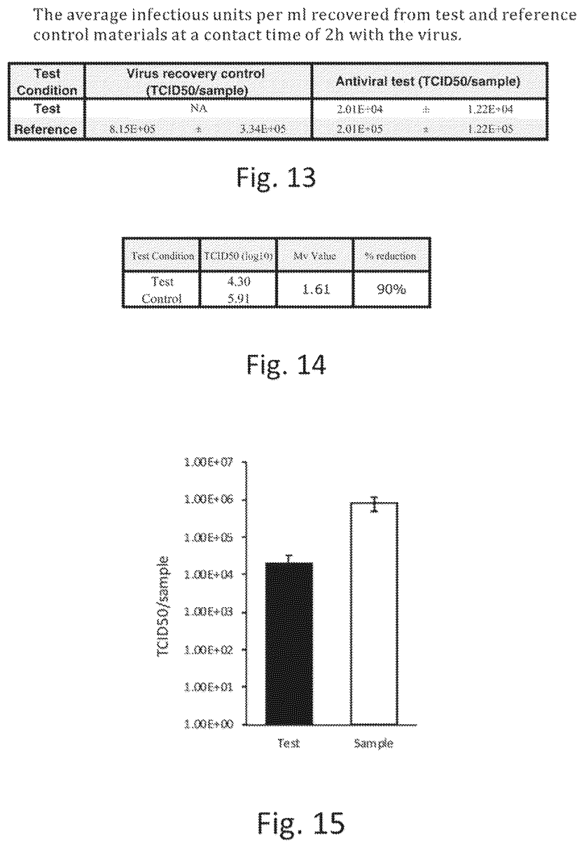

FIG. 13 is a table listing the average infectious units per ml recovered from test and reference control materials at a contact time of 2 h with SARS-CoV-2;

FIG. 14 is a table showing the antiviral activity of the treated membrane (polyester material printed with α-virion);

FIG. 15 is a graph showing the mean TDIC50/sample values for SARS-CoV-2 following a contact time of 2 h with test sample and immediately harvested from the control material;

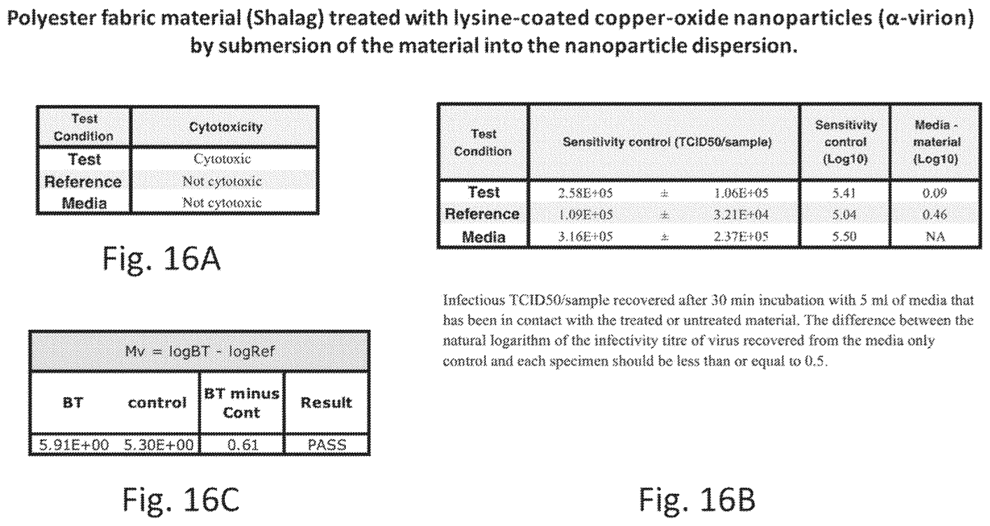

FIG. 16 shows a cytotoxicity table (A), a sensitivity control table (B) and a reduction table (C) for a polyester material treated by submersion with α-virion;

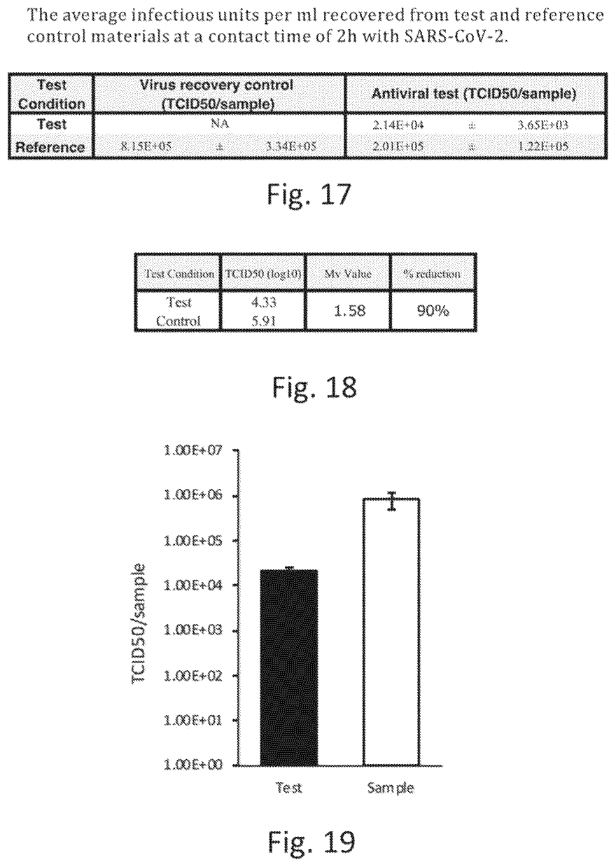

FIG. 17 is a table listing the average infectious units per ml recovered from test and reference control materials at a contact time of 2 h with SARS-CoV-2;

FIG. 18 is a table showing the antiviral activity of the treated membrane (polyester material treated by submersion with α-virion);

FIG. 19 is a graph showing the mean TDIC50/sample values for SARS-CoV-2 following a contact time of 2 h with test sample and immediately harvested from the control material;

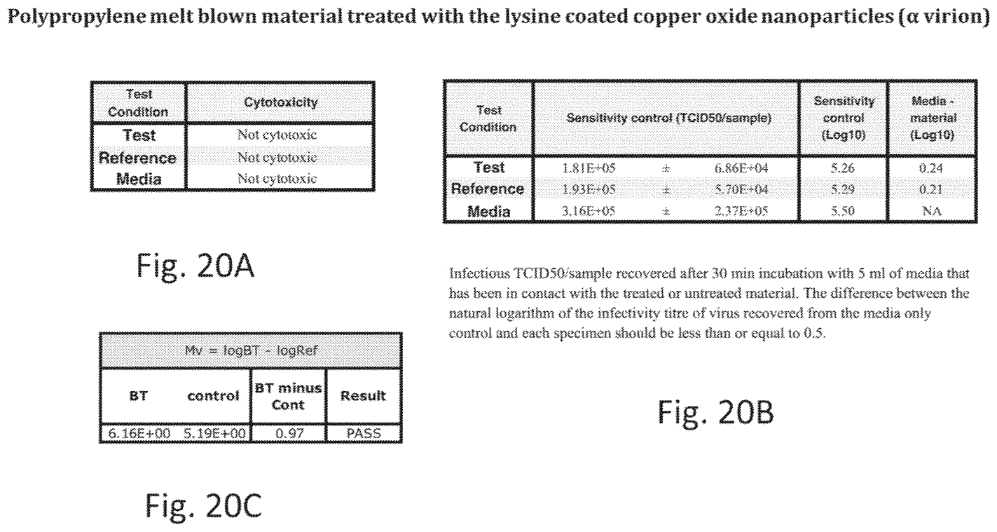

FIG. 20 shows a cytotoxicity table (A), a sensitivity control table (B) and a reduction table (C) for a polypropylene melt blown material treated with α-virion;

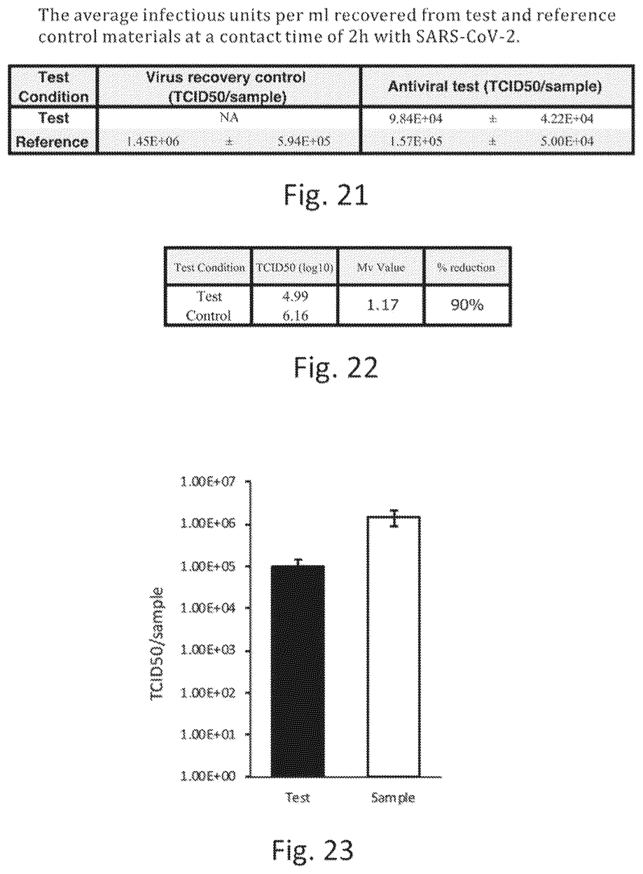

FIG. 21 is a table listing the average infectious units per ml recovered from test and reference control materials at a contact time of 2 h with SARS-CoV-2;

FIG. 22 is a table showing the antiviral activity of the treated membrane (polypropylene melt blown material treated with α-virion);

FIG. 23 is a graph showing the mean TDIC50/sample values for SARS-CoV-2 following a contact time of 2 h with test sample and immediately harvested from the control material.

FIG. 1 is schematic diagram of a spinning disc reactor (SDR) for producing liquid colloids, as described in WO2017033005A1.

The apparatus 100 comprises a first plate 101 mounted for rotation about a rotation axis 102. The first plate 101 has a reaction surface 103 with an inner portion 104 and an outer edge 105. First and second inlet lines 106, 107 are arranged to introduce respective first and second liquid feedstock materials to the reaction surface 103, the inlet lines 106, 107 extending through the plate 101. The inlet lines 106, 107 have openings arranged to allow for the feedstock materials to be introduced on to the inner portion 104 of the reaction surface 103 at or proximal to the centre of the plate 101, i.e. at or proximal to the rotation axis 102. The inlet lines 106, 107 extend along and within a rotatable axle 108 on which the first cylinder 101 is mounted. The rotatable axle 108 is mounted to a bearing 109 and is driven by a motor external to the apparatus (not shown). The inlet lines 106, 107 may be provided along a fixed inner portion within the rotatable axle 108 such that the inlet lines 106, 107 do not themselves rotate with the axle 108. Alternatively, the inlet lines 106, 107 may rotate along with the rotatable axle 108, and an interface such as a rotating union joint provided to connect each of the rotating lines with a static fluid feed line outside the vessel 110. Such an interface may alternatively be provided within the vessel adjacent to the plate, such that the inlet lines do not extend along the rotatable axle. Channels may be provided in the plate 101 to allow passage of fluids from the inlet lines 106, 107 to the reaction surface 103. Liquids may be fed through the inlet lines 106, 107, 127 by means of pumps such as peristaltic pumps (which are useful for controlled low flow rates and reduce the possibility of contamination), with a back flow regulator if required. In addition an inlet 140 may be provided through plate 101 for introduction of a fluid or a suspension of solid material such as a coating material for applying to particles produced by reaction of the first and second feedstock materials on the reaction surface 103 of plate 101.

The apparatus 100 comprises a collection unit in the form of a vessel 110 surrounding the plate 101. In this exemplary embodiment, the vessel is divided into two parts 110a, 110b, which are separable along the rotation axis 102 to allow access to the plate 101. The sealable vessel allows the plate 101 to be kept under controlled environmental conditions, such as under a controlled atmosphere. A gas inlet 111 and gas outlet 112 are provided in the vessel walls to allow gas to be introduced and withdrawn from the interior of the vessel 110. A collection port 113 is provided towards the bottom of the vessel 110 for extracting reaction products.

Although the apparatus 100 will function with only one plate 101, in preferred embodiments the apparatus comprises a second plate 121, preferably also mounted for rotation about the same rotation axis 102. In the embodiment shown, the second plate 121 is also mounted to the end of a second rotatable axle 126. As shown, the second rotatable axle 126 is mounted on a bearing 129 and driven by a motor (not shown) external to the vessel 110. A further inlet line 127 may be provided, which in the embodiment shown is within the second rotatable axle 126. The further inlet line 127 may be used, for example, to introduce a further liquid material such as a coating material for applying to particles produced by reaction of the first and second feedstock materials on the reaction surface 103 of the first plate 101. The further inlet line 127 may have an opening at or proximal to the centre of the second plate, i.e. at or proximal to the rotation axis 102. The concave conical shape of the surface 123 of the second plate 121 allows for the further liquid material to be uniformly applied to the reaction surface 103 of the first plate 101 prior to ejection of material from the outer edge 105 of the first plate 101, thereby ensuring a uniform application of material to the reaction product on the first plate 101.

A baffle 128 is preferably provided around the bearing end of the second axle 126 to direct reaction product away from the bearing and towards the outlet 113. The first and second plates 101, 121 are preferably driven by their respective motors in opposite directions, as indicated by arrows 130a, 130b, although may be driven in the same direction and/or at different speeds.

In a first operation mode, first and second feedstock materials are introduced at or near the centre of the inner portion 104 of the reaction surface 103 of the first plate 101 while the plate 101 is rotating. For example, the plate 101 may be rotating at a speed of a few hundred rpm or faster, for instance tens of thousands rpm. At such speeds, the first and second feedstock materials mix on the reaction surface 103 and form a reaction product, such as a nanoparticulate material as the materials travel along the reaction surface 103 away from the rotation axis 102. The mixture then reaches an outer portion 134 of the reaction surface 103, which in the embodiment shown is of concave conical form. This allows the further liquid material expelled from the outer edge 135 of the rotating second plate 121 to mix with the reaction product, for example by forming a coating on the nanoparticulate material, before the reaction product is expelled from the outer edge 105 of the reaction surface 103 of the first plate 101. The apparatus 100 may be oriented in use such that the reaction surface 103 of the first plate 101 faces upwards or downwards, or in any orientation in-between, depending on the particular combination of plates and liquid feedstocks used. If, for example, a coating material is to be introduced via the second plate 121, the orientation as shown in FIG. 1 may be used, since this allows for the coating liquid to be drawn outwards from the inlet line 127 and expelled from the edge 135 due to rotation of the second plate 121. The first and second liquid feedstock materials react as they pass along the reaction surface 103 of the first plate due to rotation of the first plate 101, being held on the surface 103 by wetting. As the reaction products pass towards the outer edge 105 of the first plate, the coating material ejected from the edge 135 of the second plate 121 mixes with the reaction products and produces a finished product that is then ejected from the edge 105 and on to the inner wall of the vessel 110. The orientation as shown in FIG. 1 may be more suitable when the amount of liquid material provided via the inlet line 127 is substantially greater than that provided along inlet lines 106, 107, and/or where it is important that the liquid provided along inlet line 127 does not mix until the reaction products are formed. For example, when undertaking sequential reactions such as synthesising nanoparticles followed by coating the particles, or forming soft nanoparticles followed by a curing process, the order in which the liquid feedstocks are introduced needs to be controlled. The use of a second plate has a particular advantage in that it allows for a further liquid to be introduced without causing turbulent flow that would otherwise result in some liquid leaving the first plate without the coating liquid having been applied.

By mixing the first and second feedstock materials against on the reaction surface 103—first operation mode—it is possible to minimize potential clogging of particles and therefore to produce a narrowly dispersed reaction product.

In a second operation mode, first and second feedstock materials are introduced at or near the centre of the inner portion 124 of the reaction surface 123 of the first plate 121 while the plate 121 is rotating and plate 101 is kept static. In this case the first and second feedstock materials mix on the reaction surface 123 and form a reaction product, such as a nanoparticulate material as the materials travel along the reaction surface 123 away from the rotation axis 102. The mixture then reaches an outer portion 135 of the reaction surface 123. The mixture expelled then reaches the outer portion 134 of plate 101. Plate 101 is then used for functionalising the mixture by introducing a fluid or a suspension of solid material via inlet 140. The conical shape of the inner portion of the second plate 121 helps in preventing the liquid introduced via inlet line 127 flowing off the edge of the plate and also increases the retention time while retaining a small footprint. Liquid introduced to the surface of the second plate 121 preferably only progresses to the outer edge 135 under the action of rotation of the second plate 121.

The spin disk reactor of FIG. 1 allows the manufacture of uniform sized nanoparticles at a rate of 1 kg an hour, permitting commercial scale production.

FIG. 2 is a schematic representation of a dispersible nanoparticle according to the disclosure. The dispersible nanoparticle 200 has a core 210 and a coating 220. The core 210 is made of metal or metal-alloy compound Mn, and the coating 220 includes at least one of a carboxylic acid and a water soluble polymer. The core 210 may also be combined with an oxygen based non-metallic compound Xy, to form a metal oxide or a metal hydroxide nanoparticle or a metal-alloy oxide or a metal-alloy hydroxide nanoparticle MₙX_{y}.

The metal compound may be zinc. Alternatively, the metal compound may be a rare-earth compound or a lanthanide, such as cerium (Ce). Alternatively, a metal-alloy may be used, such as copper (Cu).

The oxygen-based non-metallic compound may be oxygen itself or an anion such as a carbonate ion (CO₃⁻²), nitrate (NO₃⁻), nitrite (NO₂⁻), or hydroxide (OH⁻).

The coating 220 may be a carboxylic acid coating or a water soluble polymer coating. The coating allows the formation of monodispersed nanoparticles and prevents agglomeration when the nanoparticles are provided in suspension. The coating 220 may include an organic compound selected from one or more of an amino acid and a peptide. The organic compound preferably comprises one or more of a carboxyl, hydroxyl, and amine functional group, which function to bond the organic compound to the surface of the metal or metal-alloy core of the nanoparticle by electrostatic bonding.

Examples of carboxylic acid coatings include ascorbic acid (vitamine C) as well as non-hazardous amino acids such as lysine or histidine. Examples of water soluble polymer coatings include branched polymers, sugar polymers, hydrogel, and ethylene glycol or polyethylene glycol.

The nanoparticle 200 may be manufactured using the spinning disc reactor of FIG. 1. The coating may be performed at formation using either mode 1 or mode 2. Alternatively, the spinning disc reactor may be used to obtain the nanoparticle (core), and the coating is performed in a second step using the electrostatic coating method described in U.S. Ser. No. 10/154,628B2, by mixing the nanoparticle with the coating material to obtain a dry powder mixture. The resulting coated nanoparticles can be readily dispersed following the dry, mixing process in a solvent such as water, and the resulting dispersion can remain stable for long periods. Below are provided two examples for the manufacture of copper oxide and zinc oxide nanoparticles.

### Example 1: Copper Oxide (CuO) Nanoparticles

The synthesis of copper oxide nanoparticles requires two precursor solutions: a metal salt solution and a strong base solution. In this example the metal salt solution is copper nitrate anhydrous, 1 mol⁻¹, 187.56 g in 1 litre. The strong base solution is sodium hydroxide, 13 mol⁻¹, 520 g in 1 litre. Both precursor solutions are heated to 80° C. Each precursor solution is then fed into a reactor of the form illustrated in FIG. 1 at a feed rate of 50 ml/s via the reactant inlet lines 106 and 107 respectively.

The reactor motor is set to 500 rpm allowing the continuous formation of copper oxide nanoparticles on the reaction surface. The resultant precipitate is then collected from the outlet 113, filtered under vacuum with grade 2 filter paper, and washed with distilled water and acetone twice to aid drying. The particles are then dried in an oven at 80° C.

Once dried, the particles are coated with lysine monohydrate to functionalise the nanoparticle. The coating may be performed at formation by introducing the coating material at the inlet line 127. Alternatively coating may be performed using the electrostatic coating method described in U.S. Ser. No. 10/154,628B2, by mixing the metal oxide nanoparticle with the coating material to obtain a dry powder mixture. The dry power mixture is then dispersed in a solvent, for instance water.

### Example 2: Zinc Oxide (ZnO) Nanoparticles

Like in the previous example the synthesis of zinc oxide nanoparticles requires two precursor solutions: a metal salt solution and a strong base solution. In this example the metal salt solution is zinc chloride anhydrous 1 mol⁻¹, 136.29 g in one litre. The strong base solution is a solution of sodium hydroxide, 1 mol⁻¹, 40 g per one litre. Each precursor solution is then fed at room temperature into a reactor of the form illustrated in FIG. 1, at a feed rate of 50 ml/s via the reactant inlet lines 106 and 107 respectively. The reactor motor is set to 500 rpm allowing the continuous formation of zinc oxide nanoparticles on the reaction surface. The resultant precipitate is then collected from the outlet 113, filtered under vacuum with grade 2 filter paper, and washed with distilled water and acetone twice to aid drying. The particles are then dried in an oven at 80° C. Once dried, the particles are coated with lysine monohydrate to functionalise the nanoparticle.

It will be appreciated that other nanoparticles may be synthesized, by varying the metal salt solution and the strong base solution accordingly.

Examples of other nanoparticles may include copper, cerium oxide (CeO), cerium hydroxide (Ce(OH)₃), zinc hydroxide (Zn(OH)₂) and copper hydroxide(Cu(OH)₂), among others. These nanoparticles may also be dopped. For instance cerium may be dopped with copper to form a cerium nanoparticle doped with copper.

The average diameter of the coated nanoparticles may range between a few nanometres to several hundred of nanometres. The average particle size and uniformity of size distribution may be adjusted by varying several parameters including the rotation speed of the reactor motor, temperature, the precursor solutions' feed rates, the disc size and the disc shape. Depending on the application, the nanoparticles may be manufactured to have a size that is similar to a target pathogen. For instance, the size may be similar or smaller than a target virus. For example, the nanoparticles may be manufactured with an average diameter that is less than 100 nm, for instance about 50 nm.

Nanoparticles can be characterized for size and morphology by electron microscopy, for example transmission or scanning electron microscopy (TEM or SEM), powder x-ray diffractometry (XRD), and Fourier transform infrared spectroscopy (FTIR).

The nanoparticles as described above are dispersible in water. As a result, the nanoparticle can be suspended in an aqueous solution at a given concentration, for example molar concentration. For instance, the concentration of nanoparticles may range from about 5% to about 15%. Such an aqueous solution can be used in the manufacture of various products to inhibit a particular pathogen.

FIG. 3 is a flow chart of a method 300 for coating a membrane to be used in a product, which may be a garment, a protective device such as a face mask, or an air filter of the kind provided in an air conditioning unit, among others.

At step 310 a nanoparticle dispersion of the kind described above with reference to FIGS. 1 and 2 is provided. At step 320, a membrane is provided. At step 330 the membrane is either dipped into the dispersion or sprayed with the nanoparticle dispersion. The step 330 may be repeated several times in order to achieve the desired thickness of nanoparticle coating. In this instance, the membrane may be dried before repeating step 330. At step 340 the coated membrane is heated to bind the nanoparticles to the surface of the membrane. This annealing process may be achieved using infrared light.

By dipping or spraying the membrane, rather than incorporating the nanoparticles into a spun fibre, a coating layer is formed at the surface of the membrane. This increases the contact area between the pathogen (virus/bacteria) and the nanoparticles, therefore enhancing anti-pathogenic properties of the membrane.

In an alternative implementation of the method, the step 330 is changed by printing the dispersion onto the membrane (instead of dipping or spraying). This can be achieved using a print head loaded with the dispersion of nanoparticles.

The process 300 may be used in the production of a face mask. Conventional disposable face masks are formed of three layers of material, known as 3 ply masks: an inner layer, a middle layer and an outer layer. The inner layer is designed to be directly applied on the wearer's airways and is usually made of a soft cotton material to ensure comfort and absorbency of moisture of exhaled air. The middle layer is usually made of a non-woven material such as polypropylene that aids the entrapment of particulate matter such as bacteria and viruses. Finally, the outer layer is provided by a non-absorbent material, such as polyester or polyester blend. The three layers are folded to increase the surface area of the mask and ease inhalation and exhalation when worn.

FIG. 4 is a schematic diagram of a face mask according to the disclosure. The face mask 400 is formed of four layers or membranes (4 ply): a first membrane 410, also referred to as inner membrane, a second membrane 420 also referred to as outer membrane, and two middle membranes 430 and 440 sandwiched between the inner membrane 410 and the outer membrane 420.

The inner membrane 410 may be made of a hydrophilic material, such as cotton, to prevent passage of bacterial and viral particulates. The outer membrane 420 may be made of a hydrophobic material or non-absorbent material, to repel moisture away from the subject to decrease the passage of pathogens via water molecules. The middle membrane 430 may be made of melt blown material to entrap pathogen agents that may have been exhaled out by the subject.

The middle membrane 440, also referred to as active membrane, is coated with the metal or metal-alloy nanoparticles of the disclosure. For instance, the middle membrane 440 may be formed of a non-woven polymer membrane 446 such as a melt blown membrane treated with a nanoparticle dispersion to form the nanoparticle coating layers 442, 444 on both sides of the membrane 446.

In use the active membrane 440 is used to trap and inhibit pathogens drawn toward the subject airways as the subject breath in. The active membrane 440 is also used to trap and inhibit pathogens that may be expelled by the subject himself as the subject breath out.

In a specific example the membrane 446 is treated with a dispersion of copper oxide nanoparticles. The copper oxide coated nanoparticles are suspended in an aqueous solution at a concentration of 5-15%. The melt blown membrane 446 is then dipped into the dispersion or sprayed with the dispersion on both sides of the membrane 446. This step may be repeated several time to obtain a coating having the desired thickness of nanoparticles. For instance, the nanoparticle coating may have a thickness ranging from about 5 nm to about 60 nm. After application, infrared light is used for heating and binding the nanoparticle formulation to the nanofiber of the melt blown membrane 446. This annealing process prevents the nanoparticles from being released in the environment or from entering the airways of the subject wearing the mask. The membrane 440 is then sterilised in preparation for facemask manufacture with UV light.

Various modifications can be envisaged. For instance, the inner membrane 410 and the outer membrane 420 may be made of cellulose. It will also be appreciated that the mask would also include other parts such as a nose band and ear loops not represented in FIG. 4. These parts may be made of biodegradable material to obtain a fully biodegradable mask.

In an alternative embodiment the membrane 430 is not used.

FIG. 5 illustrates an air conditioning unit 500. The air conditioning unit includes a motor 510 for generating an air flow, and a filter 540 provided between an input 520 and an output 530. The filter 540 is made of a membrane 546 having and two nanoparticle layers 542 and 544 on each side of the membrane 546. The filter 540 may be manufactured by dipping or spraying the membrane 546 with a nanoparticle dispersion, as described above with reference to FIG. 3.

In use the motor 510 generates an air flow between the input 520 and the output 530. The air is filtered by the filter 510 which traps and inhibit at least partially certain pathogens, such a viruses and bacteria.

The nanoparticle dispersion of the present disclosure can also be used in a sanitizing product such a hand gel or a disinfecting spray. The type of nanoparticles selected may vary depending on the specific application. For instance, a mixture of different nanoparticles may be used. The disinfectant may have both anti-viral and anti-bacterial properties. A gel may be formed by mixing the nanoparticle dispersion with a polymer network. For instance, the gel may be a hydrogel. In this case the polymer will allow prolonged suspension of the nanoparticles giving the particles longer shelf life.

The nanoparticle dispersion of the present disclosure may also be used to treat clothing. For instance, the nanoparticle dispersion can be sprayed or deposited onto a piece of textile or fabric material. The nanoparticle dispersion may also be used as part of a washing process to protect the textile or fabric material against pathogens including virus, bacteria or parasites. A skilled person will therefore appreciate that variations of the disclosed arrangements are possible without departing from the disclosure.

FIG. 6 is a transmission electron microscope image of a melt blown membrane 600 treated with a dispersion of lysine-coated copper-oxide nanoparticles (CuO_Lys). The image shows CuO_Lys nanoparticles embedded onto the surface of melt blown fibres.

The antiviral activity of the melt blown membrane treated with CuO_Lys nanoparticles 600 was tested against influenza A virus at a contact time of 7 hours relative to a non-treated reference control. The study reveals that the treated membrane displays virucidal activity against influenza A virus with a reduction of infectious virus above 90%.

Methods

For the assay to be valid, the material tested must have no cytotoxic activity on the cells used to quantify the virus, nor interfere with cell sensitivity to infection. The two tests of these criteria are described below.

Cytotoxicity control: Is the tested material cytotoxic to the assay's host cells? Assay media is added to treated material and reference control for 5 min, before been collected and added onto monolayers of cells seeded into the wells of a 96-well plate. Cells are then cultured, and after 6 days a viability assay (crystal violet staining) is used to determine cell viability. The test is carried out in triplicate for both the treated material and nontreated reference control. Media that has been in contact with neither the treated material nor reference control is included as a reference. For the test to be valid, no cytotoxic effect should be observed compared to the media.

Sensitivity control: Do the tested materials affect the assay cells' sensitivity to the virus?

Assay media is added to treated material and reference control for 5 min, before being collected in tubes. Next, to test whether exposing the media to the materials affects the cells' sensitivity to infection, 1×10\hat{ }5 infectious units of virus are added into each tube. After a 30-min incubation at 25° C., the amount of infectious virus in each sample is quantified (TCID50 assay). The 50% tissue culture infectious dose (TCID50) is the end-point virus dilution where 50% of the infected test cells die.

The tests are carried out in triplicate on treated and non-treated material. Media that has not been in contact with either material is also incubated with the virus.

When there is no cytotoxicity and the materials do not interfere with the host cell's sensitivity to infection, the assay is considered to meet the requirements for 15018184 and can be used to establish the antiviral activity of the test material.

Antiviral Test Procedure

Treated and non-treated fabrics were placed in individual tubes in triplicate. A liquid volume (200 μl) of an appropriate concentration of virus (1×10\hat{ }5 PFU/ml stock of virus) was added on top of each fabric so that the material is completely imbibed with the virus solution. A lid was placed over each tube, which was then incubated for the indicated contact time at 25° C. At the end of the incubation, the samples were washed with media several times to recover the virus. The amount of infectious virus recovered from each sample was then quantified by TCID50. As a further control, virus was added to three samples of the non-treated reference control material and immediately recovered by washing (referred to as the ‘virus recovery control’ or ‘back-titration’). This recovered virus is used to quantify the starting amount of virus.

TCID50 Determination

A seven-point, ten-fold serial dilution from the virus-containing wash media was tested in quadruplicate for each sample on Madin-Darby Canine Kidney (MDCK) cells. After 6 days, a viability crystal violet assay was carried out to determine cell viability across the dilution series. The dilution at which 50% of cells are infected/killed (TCID50) was calculated using a regression analysis.

Quantification of Antiviral Activity

When the test is deemed valid, the antiviral activity (Mv) is calculated as follows: My=Ut−At where Ut is the average of the common logarithm of the number of infectious units recovered from the untreated test specimens at the end of the incubation time; and At is the average of the common logarithm of the number of infectious units recovered from the treated test specimens at the end of the incubation time. An My value of 1 indicates antiviral activity.

Results

FIG. 7 shows the cell viability upon incubation with media recovered from reference and treated materials, relative to the fresh media control.

FIG. 8 shows the infectious TCID50/ml recovered after 30 minutes incubation with 5 ml of media that has been in contact with the treated or untreated material.

The test material displays cytotoxicity towards the cells used to host the virus in this experiment. The test material does not interfere with the cells used to host the virus in this experiment. Because of cytotoxicity, the first dilution of the recovered media (the undiluted sample) was excluded from the analyses. In this way, the antiviral activity of the sample can be tested.

FIGS. 9, 10 and 11 show the results of the antiviral test. The treated material displays virucidal activity against influenza A virus when using a contact time of 7 h. The treated material displays antiviral activity against influenza A virus. FIGS. 9 and 10 show that the average recovered titre for the treated material was 1.40E+04 TCID50/ml compared to the average recovered viral titre of 3.30E+05 TCID50/ml for the non-treated reference control. As a result the antiviral activity My=1.37, as shown in FIG. 11.

Based on the above findings and following 15018184, the treated material displays virucidal activity against influenza A virus after a contact time of 7 h. The results of control assays confirm that the tested material is not cytotoxic for the test cells (when excluding the first dilution—the undiluted sample—of the recovered media). Also, the test material does not interfere with the cells' sensitivity to the virus. Thus, the experiment meets the requirements for a valid ISO18184 test.

The dispersion of lysine-coated copper-oxide nanoparticles (CuO_Lys) also referred to as α-virion has been tested on different types of non-woven materials for antiviral activity against SARS-CoV-2 using EU standard protocol for ISO18184:2019. The first non-woven material, known as Shalag, is made of polyester. The second non-woven material is a melt blown material made of polypropylene. The Shalag material was treated with the dispersion of lysine-coated copper-oxide nanoparticles (CuO_Lys) using two different processes: a) by submersion of the Shalag fabric material into the dispersion of nanoparticles or b) by printing the dispersion of nanoparticles onto the surface of the Shalag fabric material using a print head.

Methods

For the assay to be valid, the material tested must have no cytotoxic activity on the cells used to quantify the virus, nor interfere with cell sensitivity to infection. The two tests of these criteria are described below.

Cytotoxicity control: Is the tested material cytotoxic to the assay's host cells? Assay media is added to test material and reference control for 5 min, before being collected and added onto monolayers of cells seeded into the wells of a 96-well plate. Cells are then cultured, and after 3 days a viability assay (crystal violet staining) is used to determine cell viability. The test is carried out in triplicate for both the test material and nontreated reference control. Media that has been in contact with neither the test material nor reference control is included as a reference. For the test to be valid, no cytotoxic effect should be observed compared to the media.

Sensitivity control: Do the tested materials affect the assay cells' sensitivity to the virus? Assay media is added to test material and reference control for min, before being collected in tubes. Next, to test whether exposing the media to the materials affects the cells' sensitivity to infection, 0.5×10\hat{ }6 infectious units (IU) of virus are added into each tube. After a 30-min incubation at room temperature, the amount of infectious virus in each sample is quantified (TCID50 assay). The 50% tissue culture infectious dose (TCID50) is the end-point virus dilution where 50% of the infected test cells die.

The tests are carried out in triplicate on tested and reference material. Media that has not been in contact with either material is also incubated with the virus. When there is no cytotoxicity and the materials do not interfere with the host cell's sensitivity to infection, the assay is considered to meet the requirements for ISO18184 and can be used to establish the antiviral activity of the test material.

Antiviral Test Procedure

Test and control fabrics were placed in individual tubes in triplicate. 200 μl of virus (of concentration 1×10\hat{ }7 IU/ml) were added on top of each fabric so that the material was completely imbibed with the virus solution. A lid was placed over each tube, which was then incubated for the indicated contact time at room temperature. At the end of the incubation, the samples were washed with media several times to recover the virus. The amount of infectious virus recovered from each sample was then quantified by TCID50. As a further control, virus was added to three samples of the reference control material and immediately recovered by washing (referred to as the ‘virus recovery control’). This recovered virus is used to quantify the starting amount of virus. The difference between the virus recovered from the reference immediately after inoculation and after the contact time must be less than 1 log.

TCID50 Determination

An eight-point, ten-fold serial dilution from the virus-containing wash media was tested in quadruplicate for each sample on African Green Monkey Kidney Epithelial (Vero). After 3 days, a viability crystal violet assay was carried out to determine cell viability across the dilution series. The dilution at which 50% of cells are infected/killed (TCID50) was calculated using the Reed and Muench method.

Quantification of Antiviral Activity

When the test is deemed valid, the antiviral activity (Mv) is calculated as follows: My=Log(Va)−Log(Vc) where Log(Va) is the average of the common logarithm of the number of infectious units recovered from the reference specimens immediately after inoculation; and Log(Vc) is the average of the common logarithm of the number of infectious units recovered from the antiviral test specimens at the end of the incubation time.
A value of ≥2.0≥Mv 1.0 indicates mild antiviral effect.
A value of ≥3.0≥Mv 2.0 indicates good antiviral effect.
A value of Mv≥3.0 indicates excellent antiviral effect.
A) Results for the antiviral activity of the polyester material (Shalag) printed with the lysine-coated copper-oxide nano particles (α-virion).

FIG. 12A shows the cell viability upon incubation with media recovered from reference and treated materials, relative to the fresh media control.

FIG. 12B shows the infectious TCID50/ml recovered after 30 minutes incubation with 5 ml of media that has been in contact with the treated or untreated material.

The test material displays cytotoxicity towards the cells used to host the virus in this experiment. The test material does not interfere with the cells used to host the virus in this experiment. Because of cytotoxicity, the first dilution of the recovered media (the undiluted sample) was excluded from the analyses. In this way, the antiviral activity of the sample can be tested.

FIGS. 13, 14 and 15 show the results of the antiviral tests. The treated material (polyester material (Shalag) printed with the lysine-coated copper-oxide nanoparticles (α-virion)) displays virucidal activity against SARS-CoV-2 when using a contact time of 2 hours.

FIG. 13 shows that the average recovered titre for the treated material was 2.01E+04 TCID50/sample compared to the average recovered titre of 2.01E+05 TCID50/sample for the reference control (Polyester control=Shalag). As a result the antiviral activity My=1.61, as shown in FIG. 14.

Based on the findings reported here and following ISO18184, the test material displays virucidal activity against SARS-CoV-2 after a contact time of 2 hours. The results of control assays confirm that the tested material is not cytotoxic for the test cells (when excluding the first dilution—the undiluted sample—of the recovered media). Also, the test material does not interfere with the cells' sensitivity to the virus. Thus, the experiment meets the requirements for a valid ISO18184 test.
B) Results for the antiviral activity of the polyester material (Shalag) treated with the lysine-coated copper-oxide nanoparticles (α-virion) by submersion of the material into the nanoparticle dispersion.

FIG. 16A shows the cell viability upon incubation with media recovered from reference and treated materials, relative to the fresh media control.

FIG. 16B shows the infectious TCID50/sample recovered after 30 minutes incubation with 5 ml of media that has been in contact with the treated or untreated material.

The test material displays cytotoxicity towards the cells used to host the virus in this experiment. The test material does not interfere with the cells used to host the virus in this experiment. Because of cytotoxicity, the first dilution of the recovered media (the undiluted sample) was excluded from the analyses. In this way, the antiviral activity of the sample can be tested.

FIGS. 17, 18 and 19 show the results of the antiviral tests. The treated material displays virucidal activity against SARS-CoV-2 when using a contact time of 2 hours. FIG. 17 shows that the average recovered titre for the treated material was 2.14E+04 TCID50/sample compared to the average recovered titre of 2.01E+05 TCID50/sample for the reference control (Polyester control=Shalag). As a result the antiviral activity My=1.58, as shown in FIG. 18.

Based on the findings reported here and following ISO18184, the test material displays virucidal activity against SARS-CoV-2 after a contact time of 2 hours. The results of control assays confirm that the tested material is not cytotoxic for the test cells (when excluding the first dilution—the undiluted sample—of the recovered media). Also, the test material does not interfere with the cells' sensitivity to the virus. Thus, the experiment meets the requirements for a valid ISO18184 test.
C) Results for the antiviral activity of the polypropylene melt blown material treated with the lysine-coated copper-oxide nano particles (α-virion).

FIG. 20A shows the cell viability upon incubation with media recovered from reference and treated materials, relative to the fresh media control.

FIG. 20B shows the infectious TCID50/sample recovered after 30 minutes incubation with 5 ml of media that has been in contact with the treated or untreated material.

The test material does not display cytotoxicity towards the cells used to host the virus in this experiment. The test material does not interfere with the cells used to host the virus in this experiment.

FIGS. 21, 22 and 23 show the results of the antiviral tests. The treated material (melt blown material treated with the lysine-coated copper-oxide nanoparticles (α-virion)) displays virucidal activity against SARS-CoV-2 when using a contact time of 2 hours.

FIG. 21 shows that the average recovered titre for the treated material was 9.84E+04 TCID50/sample compared to the average recovered titre of 1.57E+05 TCID50/sample for the reference control (Polypropylene control=Voltz 35 gsm). As a result the antiviral activity My=1.17, as shown in FIG. 22.

Based on the findings reported here and following ISO18184, the test material displays virucidal activity against SARS-CoV-2 after a contact time of 2 hours. The results of control assays confirm that the tested material is not cytotoxic for the test cells. Also, the test material does not interfere with the cells' sensitivity to the virus. Thus, the experiment meets the requirements for a valid ISO18184 test.

The Shalag material treated with lysine-coated copper-oxide nanoparticles can be used in the fabrication of medical face masks. The face mask obtained known as Pro-Larva, has passed the conformity assessment procedure for medical face masks (EN 14683 standard). The standard tests various factors including bacterial filtration efficiency, microbial cleanliness, resistance against penetration by synthetic blood and biocompatibility and breathability. The α-virion antiviral layer shows a virucidal activity against Influenza A virus, human coronavirus OC43 and Schmallenberg virus.

The nanoparticle described in the present application (i.e comprising a core made of a metal or a metal alloy compound, in which the core is coated with at least one of a carboxylic acid and a water soluble polymer) may be suspended in an ink solution to obtain an anti-pathogenic ink solution having at least one of an anti-viral, an anti-bacterial, and an anti-parasitic property.

For instance, the copper oxide coated nanoparticles presented in the application may be suspended in an ink solution. The ink solution may be applied to a roller and then printed on paper, cardboard or other materials. This may be used to provide packaging having an anti-viral, an anti-bacterial, and an anti-parasitic property.

The nanoparticle described in the present application may also be suspended in a coating such as a paint or a wax, hence providing an antipathogenic coating that may be applied to various substrates and surfaces.

The above description of the specific embodiments is made by way of example only and not for the purposes of limitation. It will be clear to the skilled person that minor modifications may be made without significant changes to the operation described. The antiviral activity tests presented were performed by Virology Research Services Ltd, Nottingham, UK.

1-28. (canceled)

29. A dispersible nanoparticle for use in anti-pathogenic applications, comprising:
a core made of a metal alloy compound comprising copper;
wherein the core is coated with a carboxylic acid;
wherein the core is combined with an oxygen based non-metallic compound to form a copper oxide or a copper hydroxide nanoparticle; and
wherein the dispersible nanoparticle has an anti-viral property.

30. The dispersible nanoparticle as claimed in claim 29, wherein at least one of:
the oxygen based compound comprises one of a carbonate ion, a nitrate ion, a nitrite ion, and a hydroxide ion; and
the dispersible nanoparticle has a diameter of less than about 100 nm when measured using electron microscopy.

31. The dispersible nanoparticle as claimed in claim 29, wherein the carboxylic acid coating comprises:
at least one of ascorbic acid, lysine, and histidine; or
at least one of an amino acid and a fatty acid.

32. A nanoparticle dispersion comprising a concentration of dispersible nanoparticles as claimed in claim 29.

33. The nanoparticle dispersion as claimed in claim 32, wherein at least one of:
the nanoparticle dispersion is an aqueous dispersion; and
the nanoparticle dispersion comprises a polymer network to form a gel.

34. The nanoparticle dispersion as claimed in claim 32, wherein at least one of:
the concentration of nanoparticles ranges between about five percent and about fifteen percent of nanoparticles; and
the nanoparticle dispersion comprises at least one of copper, copper oxide, and copper hydroxide nanoparticles.

35. The nanoparticle dispersion as claimed in claim 32, wherein the nanoparticle dispersion is an anti-pathogenic solution having at least one of an anti-viral, an anti-bacterial, and an anti-parasitic property.

36. A membrane coated with the dispersible nanoparticles as claimed in claim 29.

37. The membrane as claimed in claim 36, wherein the membrane is treated with the nanoparticle dispersion as claimed in claim 32.

38. The membrane as claimed in claim 36, wherein the membrane is adapted to filter a volume of air.

39. The membrane as claimed in claim 36, wherein the membrane is made of a non-woven polymer material comprising a melt blown material.

40. The membrane as claimed in claim 36, wherein the membrane is an anti-pathogenic membrane having at least one of an anti-viral, an anti-bacterial, and an anti-parasitic property.

41. A product comprising a membrane as claimed in claim 36.

42. The product as claimed in claim 41, wherein the product is a face mask.

43. The product as claimed in claim 42, wherein the membrane is sandwiched between an inner layer and an outer layer.

44. The product as claimed in claim 41, wherein the product is selected from the group consisting of:
an air filter for use in an air conditioning unit, the membrane being part of the air filter; and
a piece of clothing, the membrane being made of textile or fabric material.

45. A method of coating a membrane, the method comprising providing a nanoparticle dispersion as claimed in claim 32;
providing a membrane;
performing a step selected from the group consisting of:
dipping the membrane in the dispersion;
spraying the membrane with the dispersion; and
printing the dispersion onto the membrane; and
heating the membrane to bind the nanoparticles to its surface.

46. A liquid product for marking a substrate, wherein the liquid product comprises:
a nanoparticle dispersion as claimed in claim 32; and
a colorant comprising at least one of a dye and a pigment.

47. The liquid product as claimed in claim 46, wherein the liquid product is an ink.

48. The liquid product as claimed in claim 46, wherein the liquid product is a paint.

49. The membrane as claimed in claim 39, wherein the non-woven material comprises polyester or polypropylene.

50. The product as claimed in claim 43, comprising an additional membrane provided between the inner layer and the outer layer.
